(19) United States
(12) Patent Application Publication
(10) Pub. No.: US 2019/0071473 A1
(43) Pub. Date: Mar. 7, 2019

(54) REGULATION OF SUBCELLULAR LIPID DISTRIBUTION

(71) Applicant: The Regents of the University of California, Oakland, CA (US)

(72) Inventors: Chien Yu Huang, Riverside, CA (US); Anthony H.C. Huang, Riverside, CA (US)

(21) Appl. No.: 15/980,403

(22) Filed: May 15, 2018

Related U.S. Application Data
(60) Provisional application No. 62/511,494, filed on May 26, 2017.

(51) Int. Cl. C07K 14/415; C12N 15/82; C12N 15/74

(52) U.S. Cl. CPC C07K 14/415; C12N 15/8247; C07K 2319/055; C07K 2319/60; C12N 15/74

(57) ABSTRACT

The present invention provides a modified oleosin protein, a polynucleotide sequence encoding the modified protein, and a method for regulating subcellular lipid distribution by recombinantly expressing the modified oleosin protein. This invention also provides a method for generating cells and organisms comprising the modified oleosin protein and exhibiting an altered subcellular lipid distribution pattern, as well as cells and organisms generated by such a method.

### RELATED APPLICATIONS

This application claims priority to U.S. Provisional Patent Application No. 62/511,494, filed on May 26, 2017, the contents of which are hereby incorporated by reference in the entirety for all purposes.

### REFERENCE TO SUBMISSION OF A SEQUENCE LISTING AS A TEXT FILE

The sequence listing written in file 081906-1082455i-220730us_sl.txt created on Oct. 10, 2018, 13,046 bytes, machine format IBM-PC, MS-Windows Operating System, is hereby incorporated by reference in its entirety for all purposes.

### BACKGROUND OF THE INVENTION

Cytoplasmic lipid droplets (LDs) of neutral lipids are reserves in cells of all eukaryotes and prokaryotes. Recently, studies of LDs have attracted significant attention for their involvement in the production of biodiesels in photosynthetic organisms and high-valued lipid-related products in diverse organisms, as well as in human diseases related to obesity and host-pathogen interaction. LDs in plant seeds are prominent and were studied extensively before those in other organisms. Plant LDs are covered with a layer of phospholipids and abundant structural protein called oleosin. Oleosin has short amphipathic N- and C-terminal peptides flanking a 72-residue hydrophobic hairpin, which penetrates and stabilizes the LD. Oleosin is synthesized on endoplasmic reticulum (ER) and extracts ER-budding LDs to cytosol. The present inventors examined oleosin targeting signals for ER-LDs by expressing recombinant-oleosin genes in Physcomitrella patens after transient expression and Nicotiana tabacum cells after stable transformation. The initial ˜4 residues and the entirety of the hairpin, but not the N- and C-terminal peptides, were required for oleosin targeting to ER and staying on LDs. Oleosin with additions of an N-terminal ER-targeting peptide and a vacuole-targeting propeptide and a reduction of the hairpin length entered the ER lumen; extracted ER-budding LDs to the lumen; and guided LDs to vacuoles. These findings define the mechanism of oleosin and LD biosynthesis and reveal approaches to redirecting cytosolic LDs to storage vacuoles or secretion to avoid metabolic feedback inhibition and for other industrial and health applications.

Cytoplasmic lipid droplets (LDs) of neutral lipids are reserves in all eukaryotes and prokaryotes (1-10). The spherical LDs are 1-2 μm in diameter, depending on cell types and metabolic conditions. Each LD is enclosed with a layer of phospholipids (PLs) embedded with proteins, which exert structural and/or metabolic/regulatory functions. Vegetative oils from plant LDs have been extensively used for food and non-food purposes. Recently, studies of LDs have attracted major attention because they are involved in industrial manufacture of renewable biodiesels in photosynthetic organisms (11) and high-valued lipid-related products in diverse organisms (12), as well as human diseases related to obesity and host-pathogen interactions (6-10).

LDs in plant seeds are prominent and were studied extensively (1, 2) before those in mammals and microbes (5-10). Seeds store triacylglycerols (TAGs) in LDs (also called oil bodies, oleosomes, lipid bodies, spherosomes, etc.) as food reserves for germination. Each LD has a TAG matrix enclosed with a layer of PLs and the structural protein oleosin. Oleosin completely covers the surface of LDs and prevents them from coalescing, even in desiccated seeds (2, 13). The small size of LDs provides a large surface area per unit TAG, which facilitates lipolysis during germination.

Oleosins are present in green algae and primitive and advanced plants (1, 2, 13, 14, 30). They are small proteins of 15-26 kDa. Each oleosin has short amphipathic N- and C-terminal peptides lying on the LD and a hallmark central hydrophobic hairpin of ˜72 uninterrupted non-charged residues. The hairpin has 2 arms each of ˜30 residues linked with a loop of 12 most-conserved residues (PX₅SPX₃P (SEQ ID NO:1), with X representing a nonpolar residue). The hairpin of an alpha (15) or beta (16) structure of 5-6 nm long penetrates the surface PL layer into the TAG matrix of an LD and stabilizes the whole LD.

Seed LDs are synthesized on endoplasmic reticulum (ER) (1, 2). TAG-synthesizing enzymes are associated with extended regions or subdomains of ER (17-23). TAGs synthesized on ER are sequestered in the non-polar acyl region of the PL bilayer, which results in an ER-budding LD. Oleosin is synthesized on the cytosolic side of ER via Signal-Recognition-Particle-guided mRNAs (22) and extracts ER-budding LDs to cytosol. The C-terminal peptide is not required for oleosin targeting to ER-LDs (21, 22), but the N-terminal peptide may or may not be so required (21, 24, 25). The hairpin and its loop PX₅SPX₃P (SEQ ID NO:1) are required for proper oleosin targeting to ER-LDs (21). Adding an N-terminal ER-targeting peptide to oleosin allows the protein to associate with ER but not enter the ER lumen, presumably because of the bulky hydrophobic hairpin (21).

Seed LDs produced from ER budding are discharged to and stored in cytosol, because oleosin is synthesized on the ER cytosolic side and extracts budding LDs to cytosol. If oleosin were synthesized on the ER luminal side, budding LDs could be extracted to the ER lumen and then move to protein storage vacuoles (PSVs) or other vesicular compartments, or be excreted. Plant vacuoles serve many functions (26-28), one of which is being metabolic sinks of accumulated secondary metabolites, not just for storage but also for avoidance of metabolic feedback inhibition.

Here the present inventors have delineated the mechanisms of oleosin synthesis and its targeting to ER-LDs. From the findings, they have modified oleosin with additions of an N-terminal ER targeting peptide and a vacuole-targeting propeptide and a reduction of the hairpin length; the modified oleosin enters the ER lumen and guides ER-budding LDs to the lumen and then vacuoles. The inventors have delineated the mechanisms of oleosin synthesis and its targeting to ER-LDs. In addition, they show that oleosin with additions of an N-terminal ER targeting signal peptide and a vacuole-targeting propeptide and a reduction of the hairpin length enters the ER lumen and guides ER-budding LDs to the lumen and then vacuoles (29). These approaches have potential for industrial and health applications.

### BRIEF SUMMARY OF THE INVENTION

This invention provides a modified oleosin protein, which is effective for regulating subcellular lipids storage, transportation, and disposition thus possesses potentials of substantial importance in various applications. Thus, in one aspect, the present invention relates to a recombinant or modified oleosin protein generated from a native oleosin protein. Typically, the native oleosin protein comprises an amphipathic N-terminal peptide, an amphipathic C-terminal peptide, and a hairpin in the middle comprising or consisting of a hairpin loop flanked by two hairpin arms. In contrast, the modified oleosin protein comprises (i) an endoplasmic reticulum (ER)-targeting peptide at the N-terminus of the modified oleosin protein; and (ii) a truncated hairpin arm of the native oleosin protein—at least one hairpin arm is truncated, more typically both are truncated.

In some embodiments, the modified oleosin protein further comprises a vacuole-targeting sequence, for example, a protein storage vacuoles (PSV)-targeting sequence. In some embodiments, the vacuole-targeting sequence is located between the ER-targeting peptide and the first (or closest to N-terminus) truncated hairpin arm. In some embodiments, the modified oleosin protein has one or both hairpin arms truncated, with 5-15 (e.g., 5, 6, 7, 8, 9, 10, 11, 12, 13, 14, or 15) amino acids remaining in one or each arm. The amino acids that have been deleted from a hairpin arm may have been originally located at the N-terminus or the C-terminus of the hairpin arm; or they may have been originally located in the middle of the hairpin arm, i.e., not immediately at either of the N-terminus and C-terminus of a hairpin arm but rather at least one or a few amino acids away. In some embodiments, the amphipathic N- or C-terminal peptide of the native oleosin protein is truncated or deleted. In some embodiments, the modified oleosin protein includes the initial 5-10 amino acids of the first hairpin arm, e.g., the first 5, 6, 7, 8, 9, or 10 amino acids of the first hairpin arm counting from the direction of the N-terminus. In some embodiments, the modified oleosin protein is a fusion protein further comprising an additional heterologous peptide, or a peptide from a different origin, such as a green fluorescent protein (GFP) or β-glucuronidase (GUS). In some embodiments, the modified oleosin protein is covalently linked to a detectable label, which in addition to proteins capable of generating a detectable signal may include molecules of other nature (e.g., radioisotopes or marker peptides with commercially available antibodies).

In a second aspect, this invention provides an isolated polynucleotide sequence encoding the modified oleosin protein describe herein and above (29). In some embodiments, the polynucleotide sequence is present in an expression cassette (e.g., one that is capable of directing transcription of the coding sequence) or a vector (e.g., one that is capable of self-replicating). Also provided is a host cell comprising the expression cassette or the vector. The host cell may be a prokaryotic or eukaryotic cell. In some cases, the host cell has its genomic sequence encoding the endogenous oleosin gene and/or its regulatory elements manipulated (e.g., by deletion, substitution, or insertion of nucleotide(s) to alter the genomic sequence) such that the expression level of the endogenous oleosin protein is substantially reduced or completely eliminated. Some examples of the host cells include plant cells, animal cells, fungal cells, or bacterial cells. An organism comprising such a host cell is also provided, which may be a plant, an animal, or a microbe. In essence, the organism can be of any species, including plants and animals (invertebrates or vertebrates, such as mammals including primates such as humans) and microorganisms such as fungi (e.g., yeast), algae, all prokaryotes and eukaryotes that contain lipid droplets in their cells.

In a third aspect, the present invention provides a method of regulating distribution of subcellular lipid within a cell or a method for promoting secretion of lipids from within a cell. Both methods comprise the step of introducing into the cell the polynucleotide encoding the modified oleosin protein of this invention described above and herein. Typically, the modified oleosin protein is expressed in the cell, which may be in a permanent manner or only transiently. In some cases, the method further comprises a step of manipulating the cell's genomic sequence encoding the endogenous oleosin gene and/or its regulatory elements (e.g., by deletion, substitution, or insertion of nucleotide(s) to alter the genomic sequence) such that the expression level of the endogenous oleosin protein is substantially reduced or completely eliminated. In some embodiments, the cell is within a living organism such as a plant, an animal, or a microbe. In essence, the organism can be of any species, including plants and animals (invertebrates or vertebrates, such as mammals including primates such as humans) and microorganisms such as fungi (e.g., yeast), algae, all prokaryotes and eukaryotes that contain lipid droplets in their cells.

In a fourth aspect, this invention provides a method for generating a cell with increased extracellular secretion of lipids. This method comprises introducing into the cell the polynucleotide encoding the modified oleosin protein of this invention. In some cases, the method further comprises a step of manipulating the cell's genomic sequence encoding the endogenous oleosin gene and/or its regulatory elements (e.g., by deletion, substitution, or insertion of nucleotide(s) to alter the genomic sequence) such that the expression level of the endogenous oleosin protein is substantially reduced or completely eliminated. In some embodiments, this method may further comprise a step of selecting a cell, subsequent to the introducing step, for increased extracellular secretion of lipids. Optionally, the method scheme can include an additional step of collecting secreted lipids from a cell exhibiting increased extracellular secretion of lipids. In some embodiments, the cell is within and a part of a living organism (e.g., a plant, a non-human animal, or a microbe). An organism generated by this method is also provided, which may be a plant, a non-human animal, or a microbe. In essence, the organism can be of any species, including plants and animals (invertebrates or vertebrates, such as mammals including primates such as humans) and microorganisms such as fungi (e.g., yeast), algae, all prokaryotes and eukaryotes that contain lipid droplets in their cells.

### BRIEF DESCRIPTION OF THE DRAWINGS

FIG. 1. Subcellular localization of native and recombinant oleosins in Physcomitrella cells after transient gene expression. (a) Native and recombinant oleosins of P. patens (OLE) illustrated in linear portions. The N- and C-terminal portions are amphipathic and are shown in shaded boxes. The whole hairpin of ˜72 residues is hydrophobic; its loop (PX₅SPX₃P (SEQ ID NO:1)) and the 2 arms (33 and 26 residues) are in white circles or boxes. Three sets of recombinant oleosins include those with deletion of the C- or N-terminal portion, alteration (highlighted in red) of the 4 completely conserved P, S, P, P in the loop, and reduction of the hairpin length. In the lowest subpanel, the boxed 7 represents the initial 7 residues of the hairpin arm required for ER targeting. Green circled G represents GFP. FIG. 1a discloses SEQ ID NO: 45. (b) Images of individual cells after transformation of the respective DNA constructs encoding GFP alone or native/recombinant oleosin with its C-terminus attached to GFP. Cells were transformed with the DNA constructs, and after 12 h, GFP fluorescence and Nile Red (NR, staining of LDs) were monitored with CLSM. In the merge images, a dotted line outlines the cell circumference. Bars are all 10 μm. (c) Quantification of fluorescence in different subcellular locations with Image-J. ⁺ indicates the proportion of GFP associated with LDs (stained with Nile Red) and irregular granules (determined with CLSM). The remaining proportion was mainly with cytosol (in the test of OLEΔN31) or ER (in the tests of other recombinant OLE); the proportion in these other subcellular locations could not be assigned precisely and are not shown. * p<0.05 compared with OLE by Student t test.

FIG. 2. Subcellular localization of native and N-terminus truncated oleosins of Physcomitrella and Arabidopsis in tobacco cells after stable transformation. (a) Native and N-terminus-truncated oleosins of Physcomitrella (OLE, upper panel) and Arabidopsis (AtT, lower panel) illustrated in linear portions (following those described in FIG. 1 legend). ΔN24, ΔN28, ΔN31, ΔN6 and ΔN10 indicate the number of residues deleted from the N terminus. FIG. 2a discloses SEQ ID NO: 45. (b) Images of a portion of a cell after transformation of DNA constructs encoding various oleosins with the C-termini attached to GFP. GFP fluorescence and ER-Tracker-Red (staining ER) were monitored with CLSM. Bars are all 10 μm.

FIG. 3. Subcellular localization of recombinant oleosins, with emphasis on their association with the luminal or cytosolic side of ER, in Physcomitrella cells after transient gene expression. (a) s-OLE and a hairpin-shortened oleosin (s-OLE-10) with a 21-residue N-terminal ER-targeting peptide (s) of Physcomitrella aspartic proteinase illustrated in linear portions (following those described in FIG. 1 legend). FIG. 3a discloses SEQ ID NO: 45. (b) Images of portions of a cell after transient expression of DNA constructs encoding the recombinant oleosins and then subjected to protease protection test. In the upper panel, the cell was co-transformed with DNA constructs encoding s-OLE-GFP and BIP-RFP (ER-lumen marker). In the lower panel, the cell was co-transformed with DNA constructs encoding s-OLE-GFP and s-OLE10-RFP. The transformed cells were permeated with digitonin and then digested with trypsin. GFP and RFP were monitored with CLSM. Bars are all 5 μm.

FIG. 4. Subcellular localization of recombinant-oleosin-attached LDs, with emphasis on the locations in ER, LDs, PSVs and vacuoles, in tobacco cells after stable transformation. (a) OLE, s-OLE-10 and s-p-OLE-10 illustrated in linear portions (following those described in FIG. 1 legend). s-OLE-10 has at its N-terminus a 23-residue N-terminal ER-targeting peptide (Arabidopsis CLV3). s-p-OLE-10 has at its N terminus a 21-residue N-terminal ER-targeting peptide (bean phaesolin) followed by a 12-residue PSV-targeting propeptide (p) (Castor ricin). FIG. 4a discloses SEQ ID NO: 45. (b) Images of portions of cells after transformation of DNA constructs encoding OLE and s-OLE-10 with the C-termini attached to GFP. Fluorescence of GFP and ER-Tracker-Red (staining ER) or Nile Red (NR) was monitored with CLSM. Images in columns 2 to 4 are enlarged portions (boxed) of images in column 1, and images in column 5 are enlarged portions (boxed) of the images in column 4. (c) TEM images of portions of transformed cells containing OLE or s-OLE-10, after high-pressure freezing fixation. LDs (L, clear spherical structures), mitochondria (M) and cell wall (CW) are labeled. White arrows in the s-OLE-10 cell image indicate membranous structures enclosing or adjacent to LDs; these structures are absent in the OLE cell image. (d) Images of portions of cells after transformation of DNA constructs encoding OLE and s-p-OLE-10 with the C-termini attached to GFP. Some cells were also co-transformed with a DNA construct encoding s-p-RFP (marker of PSVs). Fluorescence of GFP, RFP and Nile Red (NR) was monitored with CLSM. Images in columns 2 to 4 are enlarged portions (boxed) of images in column 1. (e) TEM images of portions of transformed cells containing OLE or s-p-OLE-10 after chemical fixation with glutaldehyde and then osmium. Chemical fixation (used in panel e) allowed for a clear distinction between PSVs (clear) and LDs (greyish) structures, whereas high-pressure freezing fixation (no osmium; used in panel c) resulted in fairly similar, clear background of PSVs and LDs but better preservation of membranes. LDs and PSVs (V) in OLE cells were not associated, but were often associated (including LDs inside vacuoles) in s-p-OLE-10 cells. Arrows indicate potential vacuole membrane. M represents mitochondrion.

FIG. 5. Sequence of Physcomitrella oleosin and its secondary structures on the surface of a lipid droplet deduced from homology modeling. Panel a shows the sequence of an oleosin of Physcomitrella patens (PpOLE1; hereafter named OLE) (SEQ ID NO:2). The N- and C-terminal portions are amphipathic and are shown in shaded boxes. The whole hairpin of ˜72 residues is hydrophobic; its loop (PX₅SPX₃P (SEQ ID NO:1)) and the 2 arms (33 and 26 residues) are in white circles or boxes. A modified oleosin with the 2 hairpin arms shortened from 33+26 to 10+10 residues is also shown. This modified oleosin also has 7 residues in the initial N-arm of the hairpin, which are required for ER targeting (described in Results). FIG. 5a also discloses SEQ ID NO: 45. Panel b reveals the secondary structures deduced from homology modeling (website: swissmodel.expasy.org/) of the native (upper panel) and modified (lower panel) OLE on the surface of a LD. Residues with nonpolar (G, A, V, I, L, M, F, Y, W), polar (S, T, N, Q, P, C) and charged side chains (R, H, K, D, E) are shown in orange, green, and blue, respectively. Locations of the most conserved 3 proline and 1 serine residues (P59, S65, P66, and P70) of the hairpin loop PX₅SPX₃P (SEQ 1D NO:1) are in red. For the PL molecules, the gray sphere indicates the phosphate head group; yellow zip-zap line represents acyl moiety; and blue and red colors mark oxygen and nitrogen atoms, respectively. The right panel shows the enlarged loop region.

FIG. 6. Morphology of Physcomitrella and tobacco cells. Panel a shows images of Physcomitrella; from left to right: a whole vegetative body; surface light microscopy view of the single-cell-layer leafy tissue after Sudan Black staining, revealing dark spheres of LDs and green particles of chloroplasts; TEM image of a cross section of a leafy cell containing large vacuoles (V), chloroplasts (P), mitochondria (M) and LDs (L); and enlarged TEM image showing 2 LDs. Panel b shows images of tobacco BY2 cells; from left to right: light microscopy of non-green cylindrical cells in a chain; TEM image of a cross section of the cell showing several large vacuoles (V) and an LD (arrow); and enlarged TEM image showing an LD. Note that a tobacco cell is about 5× the size of a Physcomitrella cell.

FIG. 7. Subcellular localization of recombinant oleosins in Physcomitrella cells after transient gene expression. Panel a illustrates in linear portions (following those described in FIG. 5 legend) wild-type and various hairpin-shortened oleosins. All oleosins have an attachment of a 21-residue N-terminal ER-targeting peptide of Physcomitrella aspartic proteinase. FIG. 7a discloses SEQ ID NO: 45. Panel b shows images of cells after transient expression of the respective DNA constructs encoding recombinant oleosins. After transformation, GFP fluorescence (shown in green) and chloroplast autofluorescence (red) were monitored with CLSM. Bars are all 10 μm.

### DEFINITIONS

The term “oleosin protein,” as used herein, refers to small proteins of 15-26 kDa that are present in green algae and primitive and advanced plants. Each oleosin has short amphipathic N- and C-terminal peptides orienting horizontally on the LD surface and a characteristic central hydrophobic hairpin of ˜72 uninterrupted non-charged residues. The hairpin has 2 arms each of ˜30 residues linked with a loop of the 12 most-conserved residues (PX₅SPX₃P (SEQ ID NO:1), with X representing a nonpolar residue). The hairpin of an alpha or beta structure of 5-6 nm long penetrates the surface PL layer into the TAG matrix of an LD and stabilizes the whole LD. The inventor's group cloned the first oleosin gene and published the finding in 1987 and soon afterward christened the protein name oleosin. Three reviews on oleosins subsequently published are: Huang, A H C. 1992 Oil bodies and oleosins in seeds. Annu. Rev. Plant Physiol. Mol. Biol. 43, 177-200; Huang A H C. 2010 Subcellular lipid droplets and oleosins in plants. Am. Oil Chem Soc. Library on lipids (website: lipidlibrary.aocs.org/plantbio/oilbodies/index.htm); and Huang A H C. 2018 Plant lipid droplets and their associated proteins: potential for rapid advances. Plant Physiol 176: 1894-1918.

As used herein, a “signal peptide” refers to a short (5-30 amino acids long) peptide present at the N-terminus of the majority of newly synthesized proteins that are destined towards the secretory pathway. These proteins include those that reside either inside certain organelles (the endoplasmic reticulum or ER, Golgi or endosomes), secreted from the cell, or inserted into most cellular membranes. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved. A signal peptide is sometimes also referred to as signal sequence, targeting signals, localization signals, localization sequence, transit peptides leader sequence or leader peptide.

The N-terminus is the first part of the protein that exits the ribosome during protein biosynthesis. It often contains signal peptide sequences, “intracellular postal codes” that direct delivery of the protein to the proper organelle (in the current case: the organelle is ER). The signal peptide is typically removed at the destination by a signal peptidase. The N-terminal signal peptide is recognized by the signal recognition particle (SRP) and results in the targeting of the protein to the secretory pathway. In eukaryotic cells, these proteins are synthesized at the rough endoplasmic reticulum. In prokaryotic cells, the proteins are exported across the cell membrane. The N-terminal peptide from one species often works in another species. In this study, the inventors used successfully either (i) the 21-amino-acid N-terminal ER targeting sequence encoding aspartic proteinase in Physcomitrella (a primitive plant called moss) or (ii) the 23-amino-acid N-terminal ER targeting sequence encoding a protein called CLV3 in Arabidopsis (an advanced plant). For a review of signal peptides, see, e.g., Nilsson I, Lara P, Hessa T. et al. 2015 The code for directing proteins for translocation across ER membrane: SRP cotranslationally recognizes specific features of a signal sequence. J Mol Biol 427: 1191-1201.

As used herein, a “vacuole-targeting sequence” refers to a type of signal peptide specifically for targeting proteins to the subcellular vacuoles. The sequence (peptide) is located usually (but not absolutely) immediately after the N-terminal ER-targeting sequence; it is called a propeptide. The vacuole targeting peptide from one species often works in another species. In this study, the sequence (peptide) used was that (12 amino acids) for targeting a protein called ricin to subcellular vacuoles in castor bean (an advanced plant). For reviews, see, e.g., Chrispeels M J, Raikhel R V. 1992. Short peptide domains target proteins to plant vacuoles. Cell 68 (4): 613-616; Martinoia E, Meyer S, De Angeli A, & Nagy R (2012) Vacuolar transporters in their physiological context. Annual review of plant biology 63:183-213; and Zhang C, Hicks G R, & Raikhel Nev. (2014) Plant vacuole morphology and vacuolar trafficking. Frontiers in plant science 5:476.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a modified oleosin protein sequence of this invention (or a portion thereof, e.g., the hairpin loop section of a modified oleosin protein) has at least 80% identity, preferably 85%, 90%, 91%, 92%, 93, 94%, 95%, 96%, 97%, 98%, 99%, or 100% identity, to a reference sequence, e.g., a wild-type or native oleosin protein or the corresponding portion therefore, such as the hairpin loop section of a wild-type oleosin protein), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length Win the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

In the context of a fusion protein comprising a first polypeptide segment and a second “heterologous” polypeptide segment, the term “heterologous” refers to the fact that the two polypeptide segments originate from two distinct sources, for example, from two different proteins, or from the same protein but two different portions of the protein. In other words, a polypeptide being a component in a fusion protein is “heterologous” to another polypeptide component of the fusion protein when the two polypeptides do not appear in nature in the same manner as they appear in the fusion protein. The term “heterologous” has a similar meaning when used in the context of describing the relationship between two polynucleotide sequences joined together in a manner not found in nature.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

### DETAILED DESCRIPTION OF THE INVENTION

### I. General

Based on the present inventors' discovery of oleosin's important role in cellular lipid distribution, this disclosure provides a recombinant or modified protein based on a naturally occurring oleosin protein, a polynucleotide sequence encoding the protein and associated vectors, expression cassettes, and host cells, as well as methods of making and using the modified oleosin protein to modulate lipid storage/secretion at a cellular level.

### II. Production of Modified Oleosin Proteins

A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a gene of interest, such as an oleosin gene, a polynucleotide encoding a modified oleosin protein, and synthetic oligonucleotides can be verified after cloning or subcloning using, e.g., the chain termination method for sequencing double-stranded templates of Wallace et al., Gene 16: 21-26 (1981).

B. Coding Sequence for an Oleosin Protein

Polynucleotide sequences encoding a protein of interest, such as a naturally occurring oleosin protein, are typically known and in some cases may be obtained from a commercial supplier.

The rapid progress in the studies of the genome of human or other species has made possible a cloning approach where a genomic DNA sequence database can be searched for any gene segment that has a certain percentage of sequence homology to a known nucleotide sequence, such as one encoding a previously identified oleosin protein. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a cDNA or genomic library using a synthetic probe may be necessary to obtain a larger gene.

Alternatively, a nucleic acid sequence encoding a naturally occurring oleosin protein can be isolated from a cDNA or genomic DNA library of human or another species using standard cloning techniques such as polymerase chain reaction (PCR), where homology-based primers can often be derived from a known nucleic acid sequence encoding an oleosin protein. Most commonly used techniques for this purpose are described in standard texts, e.g., Sambrook and Russell, supra.

cDNA libraries suitable for obtaining a coding sequence for a naturally occurring oleosin protein may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the oleosin protein from the cDNA library. A general description of appropriate procedures can be found in Sambrook and Russell, supra.

A similar procedure can be followed to obtain a full-length sequence encoding a naturally occurring oleosin protein from a genomic library. Human genomic libraries, for example, are commercially available or can be constructed according to various art-recognized methods. In general, to construct a genomic library, the DNA is first extracted from a tissue where an oleosin protein is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding an oleosin protein is obtained.

Upon acquiring a nucleic acid sequence encoding a naturally occurring oleosin protein, the coding sequence can be further modified by a number of well-known techniques such as restriction endonuclease digestion, PCR, and PCR-related methods to generate coding sequences for oleosin proteins, including mutants and variants derived from the wild-type oleosin protein. The polynucleotide sequence encoding the desired polypeptide, e.g., a modified oleosin protein as described herein, can then be subcloned into a vector, for instance, an expression vector, so that a recombinant polypeptide can be produced from the resulting construct. Further modifications to the coding sequence, e.g., nucleotide substitutions, may be subsequently made to alter the characteristics of the polypeptide.

A variety of mutation-generating protocols are established and described in the art, and can be readily used to modify a polynucleotide sequence encoding a naturally occurring oleosin protein. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortie, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

C. Modification of Nucleic Acids for Preferred Codon Usage in a Host Organism

The polynucleotide sequence encoding a protein of interest, e.g., a modified oleosin protein, can be further altered to coincide with the preferred codon usage of a particular host. For example, the preferred codon usage of one strain of bacterial cells can be used to derive a polynucleotide that encodes a recombinant polypeptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of a protein of interest, such as a modified oleosin protein described herein.

### III. Expression and Purification of Modified Oleosin Proteins

Following verification of the coding sequence, a protein of the interest (e.g., a modified oleosin protein) can be produced using routine techniques in the field of recombinant genetics, relying on the polynucleotide sequences encoding the polypeptide disclosed herein.

A. Expression Systems

To obtain high level expression of a nucleic acid encoding a modified oleosin protein of this invention, one typically subclones a polynucleotide encoding the protein in the correct reading frame into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells (including human cells), yeast, and insect cells are well known in the art and are also commercially available. In one embodiment, the eukaryotic expression vector is an adenoviral vector, an adeno-associated vector, or a retroviral vector.

The promoter used to direct expression of a heterologous coding sequence (e.g., one encoding a modified oleosin protein) depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the modified oleosin protein of this invention in host cells. A typical expression cassette thus contains a promoter operably linked to the nucleic acid sequence encoding the modified protein and signals required for efficient polyadenylation of the transcript, ribosome binding sites, and translation termination. The nucleic acid sequence encoding the modified protein may be linked to a cleavable signal peptide sequence to promote secretion of the polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding a protein of interest (e.g., the modified olsoein protein) under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a modified oleosin protein of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

A person skilled in the art will recognize that various conservative substitutions can be made to any wild-type or mutant/variant protein to produce a modified oleosin protein within the scope of this disclosure. Moreover, modifications of a polynucleotide coding sequence may also be made to accommodate preferred codon usage in a particular expression host without altering the resulting amino acid sequence.

B. Transfection Methods

Standard transfection methods are used to produce bacterial, mammalian, yeast, insect, or plant cell lines that express large quantities of a modified oleosin protein of this invention, which can then be purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the modified oleosin protein of this invention.

In some cases, the host cell into which the modified oleosin coding sequence is being introduced may also have its genomic sequence(s) modified so as to reduce or abolish the expression of its native oleosin protein. Methods such as sequence homology-based gene disruption methods utilizing a viral vector or CRISPR system can be used for altering the oleosin genomic sequence, for example, by insertion, deletion, or substitution, which may occur in the coding region of the gene or in the non-coding regions (e.g., promoter region or other regulatory region) and which may result in substantial suppression or complete abolition of endogenous oleosin expression.

C. Purification of Recombinantly Produced Proteins

Once the expression of a recombinant protein, such as a modified oleosin protein of this invention, in transfected host cells is confirmed, e.g., via an immunoassay such as Western blotting assay, the host cells are then cultured in an appropriate scale for the purpose of purifying the recombinant protein.

1. Purification of Recombinantly Produced Polypeptides from Bacteria

When the modified proteins of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Additional methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a modified oleosin protein, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

When a recombinant polypeptide of the present invention, e.g., a modified oleosin protein, is expressed in host cells (such as human cells) in a soluble form, its purification can follow the standard protein purification procedure described below. This standard purification procedure is also suitable for purifying recombinant proteins obtained from chemical synthesis.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a modified oleosin protein of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a modified oleosin protein. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a modified oleosin protein of the present invention) can also be separated from other proteins on the basis of their size, net surface charge, hydrophobicity, or affinity for ligands, such as amylose. In addition, antibodies raised against a segment of the protein of interest (e.g., a modified oleosin protein) can be conjugated to column matrices and the target fusion protein can therefore be immunopurified. All of these methods are well known in the art.

Optionally, a cleavage site recognized by a protease may be designed into the coding sequence of the protein of this invention. For example, a cleavage site can be built in the sequence or sequences linking the target protein (e.g., a modified oleosin protein) and one or more affinity tags such as MBP or GST tag(s), such that the tag(s) can be readily removed after protease treatment.

It will be apparent to one of skill that chromatographic techniques can be performed at any scale and using equipment from many different manufacturers (e.g., Pharmacia Biotech).

### EXAMPLES

The following examples are provided by way of illustration only and not by way of limitation. Those of skill in the art will readily recognize a variety of non-critical parameters that could be changed or modified to yield essentially the same or similar results.

### Results and Discussion

Oleosin has a Mushroom- or T-Shaped Structure on a Lipid Droplet.

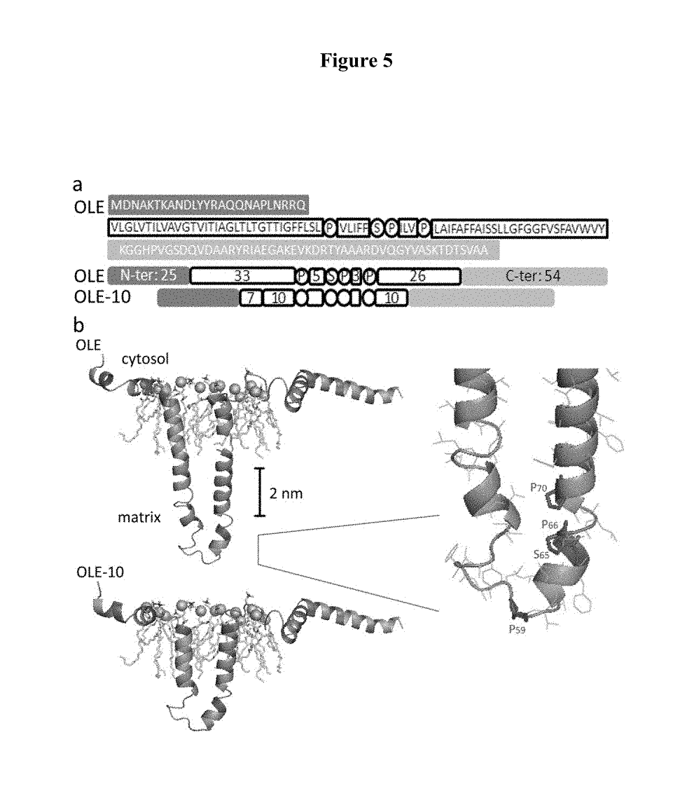

An oleosin molecule on a lipid droplet (LD) has its N- and C-terminal amphipathic peptides lying on the surface and interacting with the phospholipid (PL) charged/polar moieties, and its central hydrophobic polypeptide of ˜72 residues forming a hairpin structure and penetrating the TAG matrix (1, 2). The 2 hairpin arms could be an alpha-helix (15) or a beta-sheet structure (16). Homology modeling was used to delineate the secondary structures of oleosin (PpOLE1 from Physcomitrella [30]); hereafter named OLE) and found a mushroom- or T-shaped oleosin with the hairpin arms largely configuring an alpha-helix structure (FIG. 5). Regardless of its being an alpha or a beta structure, the hairpin is about 5-6 nm long. With this oleosin structure, the present inventors probed the signals in oleosin for targeting to ER-LDs.

N- and C-Terminal Portions of Oleosin are not Essential for Oleosin Targeting to ER-LDs.

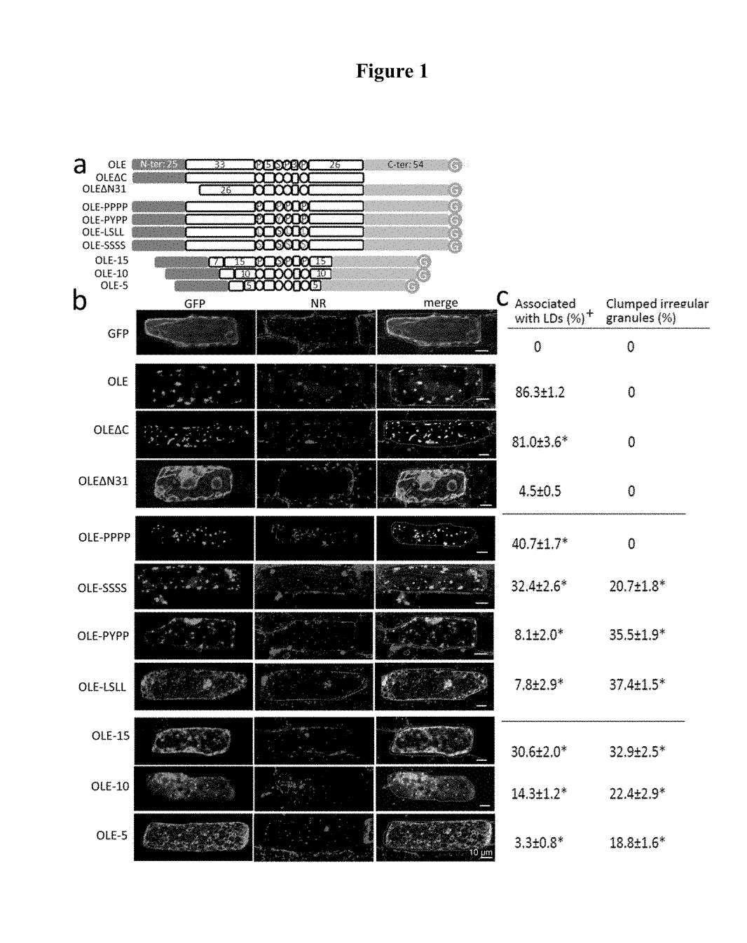

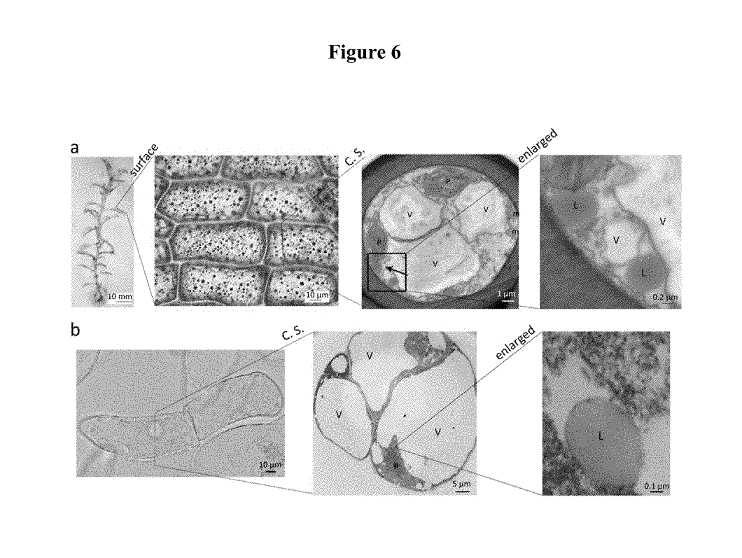

OLE was modified (FIG. 1a) to test the signals in the protein for targeting to ER and then moving onto budding LDs. Green Fluorescence Protein (GFP) was attached to the C-terminus for confocal laser scanning microscopy (CLSM). Such an attachment of GFP (30) or β-glucuronidase (GUS) (31) had no appreciable effect on oleosin targeting to LDs in vivo. DNA constructs encoding native and recombinant oleosins were transferred into the moss Physcomitrella for transient expression. The main vegetative, gametophyte body of Physcomitrella consists of branches of one-cell-layer, leaf-like tissue (FIG. 6). Each cell has several large vacuoles at the center, occupying the bulk of the cytoplasm. Most of the cytoplasm locates near the plasma membrane, with some in the inter-vacuole spaces, and contains chloroplasts of ˜5 μm and LDs of 0.5-2.0 μm in diameter.

In transformed Physcomitrella cells (FIG. 1b), free GFP (shown in green) was not associated with LDs (stained with Nile Red; red) but, rather, scattered in the cytoplasm. In contrast, OLE-GFP (green) co-located with LDs (red) (yellow droplets in merge images). A small fraction (˜10%) of OLE-GFP was associated with a putative ER network. This observation was made 16 h after transformation; at a shorter duration, more OLE-GFP was associated with ER (30), as expected because OLE-GFP targeted initially to ER. OLE-GFP without the C-portion (OLEΔC-GFP; green) also co-located with LDs (red) (yellow droplets in merge images; FIG. 1b). OLE-GPF without the 25-residue N-portion and 6 initial residues of the hairpin (OLEΔN31-GFP; green) was not associated with LDs (red) but, rather, scattered in the cytoplasm. The association of recombinant OLEs with LDs was 0%, 86%, 81% and 4% for GFP alone, OLE-GFP, OLEΔC-GFP and OLEΔN31-GFP, respectively (FIG. 1c).

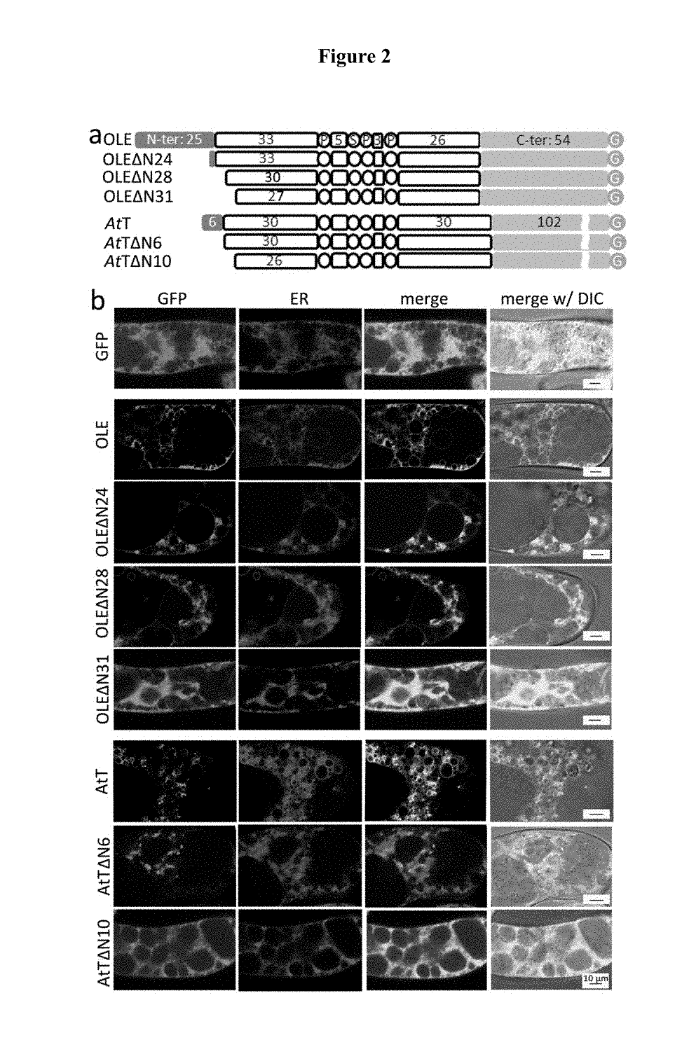

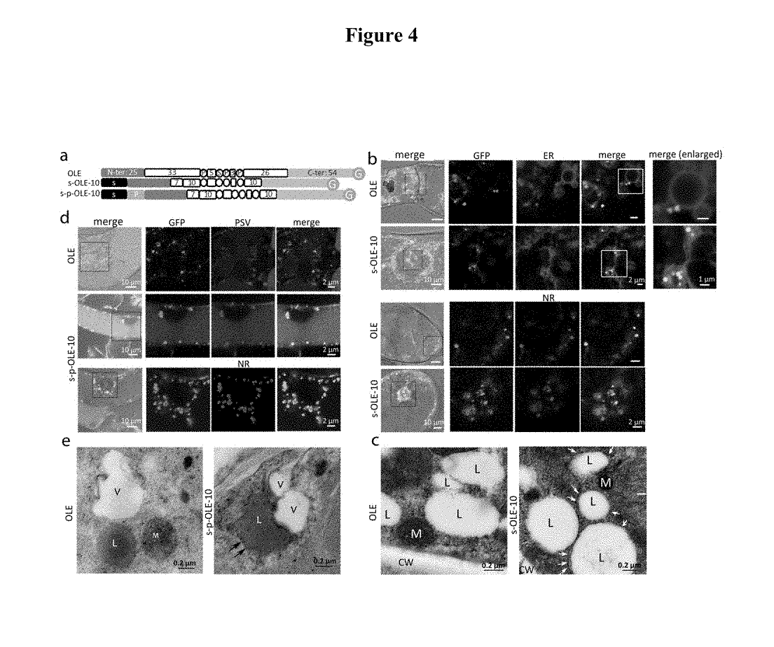

Because of the uncertainty of whether the N-portion of oleosin is (25) or is not (21, 22, 31, 32) needed for ER targeting, the N-portion (24 of the 25 residues) and the initial 3 or 6 residues of the hairpin polypeptide were deleted (OLE-GFP [wild type], OLEΔN24-GFP, OLEΔN28-GFP and OLEΔN31-GFP, respectively; FIG. 2a). To determine whether the results would apply to advanced plants, the DNA constructs encoding these recombinant oleosins were transferred into tobacco BY2 cells. Cells were subjected to stable transformation with Agrobacterium and cultured for several generations before CLSM, so that the recombinant oleosins would be produced before the tobacco internal oleosins would become dominant. The inventors determined whether the recombinant oleosins were associated with ER-LDs in transformed cells (FIG. 2b). Free GFP (green) was present throughout the cytoplasm, intermingling with the abundant ER (stained with ER-Tracker-Red; red). OLE-GFP, OLEΔN24-GFP and OLEΔN28-GFP (green) appeared largely as droplets and minimally as a network and co-located with the ER marker (red) (yellow structures in merge images; FIG. 2b). The droplets were solitary or ER-budding LDs because they were stained with Nile Red (to be shown in FIG. 4b). In contrast, OLEΔN31-GFP did not appear in droplets or a network but, rather, was scattered in the cytoplasm, similar to free GFP (FIG. 2b).

The inventors expanded the N-portion studies to an oleosin of Arabidopsis. Arabidopsis thaliana has 17 oleosins (2), and the one with the shortest N-portion (6-residue N-portion+72-residue hairpin+28-residue C-portion; FIG. 2a) was selected. This oleosin was termed AtOLE-T5 (29) (hereafter termed AtT). In transformed tobacco cells, both AtT-GFP (wild-type) and AtTΔN6-GFP (the 6-residue N-portion deleted) appeared largely as droplets and co-located with ER (stained with ER-Tracker-Red; red) (co-located structures being yellow in merge images). In contrast, AtTΔN10-GFP (the 6-residue N-portion and additional 4 residues of the hairpin deleted) was scattered in the cytoplasm, intermingling with LDs and ER. Thus, Arabidopsis and Physcomitrella oleosins were similar in that the initial hairpin residues but not the N-portion per se are required for oleosin targeting to ER-LDs.

Overall, several initial hairpin residues adjacent to the N-portion, but not the N-portion per se, of oleosin are required for the targeting. The several residues of Physcomitrella (NRRQ-VLGL) (SEQ ID NO: 43) and Arabidopsis (EIIQ-AVFS) (SEQ ID NO: 44) oleosins at the junction of the N-portion and the hairpin have no appreciable common denominators. The residues at this junction for oleosin targeting to ER-LDs require further study.

The Highly Conserved PSPP Residues of the Hairpin Loop PX₅SPX₃P (SEQ ID NO:1) of Oleosin are Required for Oleosin Targeting to ER-LDs.

The 12-residue loop, PX₅SPX₃P (SEQ ID NO:1), of the oleosin hairpin is highly conserved, and the 3 proline and 1 serine residues (PSPP in discontinuity) are completely conserved among all known oleosins (1, 2). Replacing PSPP with LLLL allows for limited targeting of the modified oleosin to ER-LDs (32). The 4 residues were modified from PSPP to PPPP, SSSS, PYPP and LSLL via the encoded genes and transferred the mutated genes into Physcomitrella (FIG. 1a). The substitutions reduced targeting of the recombinant oleosin to LDs in transformed cells from 86% (wild-type) to 41%, 32%, 8% and 8%, respectively (FIGS. 1b and c). A portion of the oleosin molecules not associated with LDs appeared as clumped granules in cytosol, apparently because of the hydrophobicity of the oleosin hairpin, and the remaining portion was scattered in the cytoplasm (FIG. 1b). Conceptually, the turn of the hairpin necessitates only 1 proline residue. Thus, the other 2 proline residues and the adjacent serine residue of PSPP could interact among themselves or with other LD ingredients for other structural or functional purposes (FIG. 5b). The small serine residue, which could not be substituted with a bulky tyrosine (both having a hydroxyl group), could be needed to form a rigid loop structure because of its hydroxyl moiety and small size.

Oleosin with a Shortened Hairpin Shows Reduced Targeting to ER-LDs.

The inventors maintained the N- and C-portions and the hairpin loop of oleosin but shortened each of the 2 hairpin arms from ˜30 (wild type) to 15, 10 and 5 residues via their encoded genes (termed OLE-GFP [wild-type], OLE-15-GFP, OLE-10-GFP and OLE-5-GFP, respectively). In transformed Physcomitrella, the recombinant oleosins showed progressive reduced targeting to LDs in proportion to the hairpin length, from 86% (wild-type) to 31%, 14% and 3%, respectively (FIGS. 1b and c). Recombinant oleosins with a shortened hairpin not associated with LDs were with an apparent ER network or present as clumped granules.

Oleosin with an Added N-Terminal ER-Targeting Peptide and a Shortened Hairpin Enters the ER Lumen.

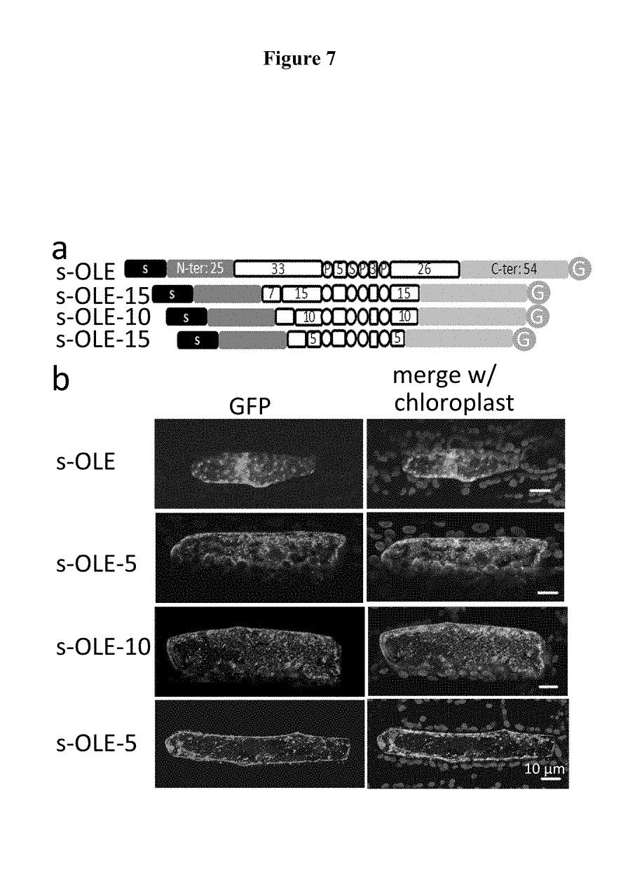

OLE was modified by adding an N-terminal ER-targeting 21-residue peptide (of Physcomitrella aspartic proteinase [33]) to the N-terminus and shortening each of the 2 hairpin arms from ˜30 (wild type) to 15, 10 and 5 residues (termed s-OLE-GFP, s-OLE-15-GFP, s-OLE-10-GFP and s-OLE-5-GFP, respectively) (FIG. 7a). In transformed Physcomitrella cells, most of the recombinant protein appeared as a network (assumed to be ER) and clumped granules. Although these subcellular structures appeared to be similar to those of recombinant oleosins with a shortened hairpin (OLE-15-GFP, OLE-10-GFP and OLE-5-GFP) but without the addition of an N-terminal ER-targeting peptide (FIG. 1b), their subcellular topologies (with or without s-attachment) were likely different. This was explored with a fluorescence protease protection assay, which involves permeating the plasma membrane but not ER membrane with the mild detergent digitonin and then applying trypsin to hydrolyze proteins in cytosol and on the ER membrane facing cytosol (34). s-OLE-GFP was used as a control and selected s-OLE-10-GFP for exploration, testing the assumption that s-OLE-GFP with its bulky hydrophobic hairpin would not (31), whereas s-OLE-10-GFP with a shortened hairpin would, enter the ER lumen.

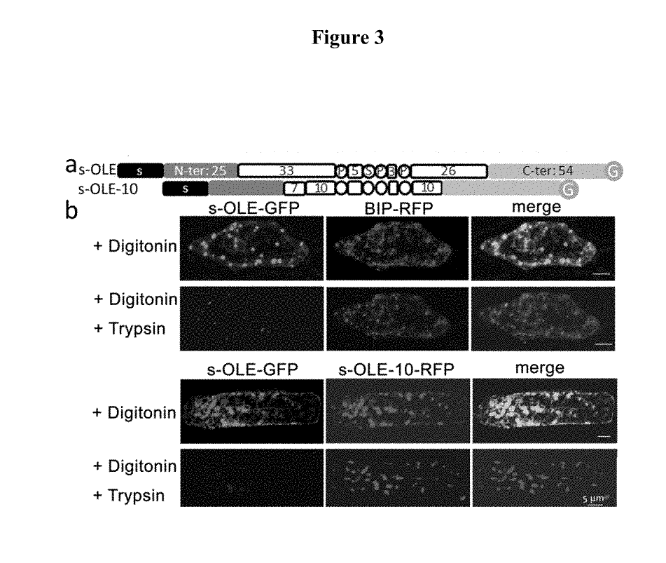

Physcomitrella cells were transformed with DNA constructs encoding s-OLE-GFP, s-OLE-10-GFP (FIG. 3a) and/or Binding Immunoglobulin Protein-Red Fluorescence Protein (BIP-RFP, an ER lumen marker [35]). In cells treated with digitonin (FIG. 3b, upper set of images), s-OLE-GFP (green) was present mostly in droplets (presumably largely solitary LDs and some ER-budding LDs) and minimally in a network (presumably ER). BIP-RFP (red) appeared in both droplets (presumably ER-budding LDs) and an ER network. The droplets and network of s-OLE-GFP and those of BIP-RFP overlapped minimally. After additional treatment of the cells with trypsin, the s-OLE-GFP-associated droplets and network disappeared, whereas the BIP-RFP-associated structures remained unchanged. Therefore, s-OLE-GFP (at least its C-terminal GFP) was present on budding LDs and ER subdomains facing cytosol, whereas BIP-RFP (at least its C-terminal RFP) was in the ER lumen. In a parallel experiment, Physcomitrella cells were co-transformed with DNA constructs encoding both s-OLE-GFP and s-OLE-10-RFP and then treated with digitonin. In the cells, s-OLE-GFP (green) appeared largely as droplets and minimally as a network, whereas s-OLE-10-RFP was present mostly as droplets (FIG. 3b, lower set of images). The s-OLE-GFP and s-OLE-10-RFP droplets co-located (yellow in merge images). After the cells had been further treated with trypsin, s-OLE-GFP disappeared whereas s-OLE-10-RFP remained unchanged. Thus, s-OLE-GFP (at least its C-terminal GFP) faced cytosol, whereas s-OLE-10-RFP (at least the C-terminal RFP) was in the ER lumen. The droplets in cells with s-OLE-GFP and s-OLE-10-RFP (FIG. 3b, lower set of images) were ˜2 times larger than those in cells with S-OLE-GFP and BIP-RFP (FIG. 3b, upper set of images) and had a relatively non-spherical shape. These larger and non-spherical-shaped LDs were interpreted as fused or continuously enlarging budding LDs on ER without budding off as a result of competing forces of s-OLE-GFP pulling from the cytosolic side and s-OLE-10-RFP pulling from the luminal side.

Oleosin with an Added N-Terminal ER-Targeting Peptide and a Shortened Hairpin Directs Budding LDs into the ER Lumen.

Because s-OLE-10-GFP successfully entered the ER lumen, the inventors tested whether the luminal s-OLE-10-GFP could extract ER-budding LDs to the luminal rather than cytosolic side. The Physcomitrella transient expression system was not used, because the cells would already have had native oleosin-coated solitary LDs in or ER-budding LDs facing cytosol. Instead, tobacco cells were used for stable transformation and the transformed cells were grown for several generations, such that s-OLE-10-GFP would outcompete native oleosin and extract ER-budding LDs into the ER lumen. In tobacco cells transformed with a DNA construct encoding OLE-GFP or s-OLE-10-GFP (FIG. 4b, upper set of images), OLE-GFP (green) appeared mostly as droplets in cytosol, and those associated with ER (stained with ER-Tracker-Red; red) were on the ER surface rather than interior. s-OLE-10-GFP also appeared mostly as droplets (green) but located inside swollen ER structures (red) (yellow droplets in merge images). The droplets with OLE-GFP or s-OLE-10-GFP were LDs because they stained positively with Nile Red (red) (FIG. 4b, lower set of images). Transmission electron microscopy (TEM) revealed that LDs in cells with s-OLE-10-GFP but not cells with OLE-GFP had enclosing or adjacent membranes (FIG. 4c), which agrees with CLSM findings that the s-OLE-10-GFP-associated LDs were present inside the ER lumen.

Modified Oleosin (s-OLE-10-GFP) with a Further Addition of a Vacuole-Targeting Propeptide Directs ER-Luminal LDs to Vacuoles.

Because s-OLE-10-GFP extracted LDs to the ER lumen, the inventors tested whether these luminal LDs firmly bonded to the ER luminal surface or were held in the lumen because of their bulkiness or whether they could be exported to the cellular exterior via a default pathway or moved to PSVs. They did not observe the export of s-OLE-10-GFP-associated LDs to the cellular exterior of transformed tobacco cells. Therefore, the inventors examined whether s-OLE-10-GFP attached to a PSV-targeting propeptide would guide s-OLE-10-GFP-associated LDs in the ER lumen to PSVs.

The present inventors made a DNA construct encoding s-p-OLE-10-GFP that included a 12-residue PSV-targeting propeptide (of castor ricin [36]) (FIG. 4a). DNA constructs encoding OLE-GFP or s-p-OLE-10-GFP and s-p-RFP (no OLE; a PSV marker) were co-transferred into tobacco cells. In transformed cells, the control OLE-GFP appeared in droplets (green) in the cytoplasm (first row, FIG. 4d) independent of PSVs and large vacuoles (s-p-RFP; red) (see TEM images of cells in FIG. 6). In contrast, s-p-OLE-10-GFP-associated LDs (green; second row, FIG. 4d) co-located with PSVs and large vacuoles (RFP; red) (yellow structures in merge images). The s-p-OLE-10-GFP-associated droplets (green) were LDs, which stained positively with Nile Red (third row, FIG. 4d) (yellow droplets in merge images). In OLE-GFP transformed cells (FIG. 4d, first row, left image), the very large vacuoles were red because they contained s-p-RFP (red) and no OLE-GFP (green); in s-p-OLE-10-GFP transformed cells (FIG. 4d, second row, left image), the very large vacuoles were greenish yellow because they contained both s-p-RFP (red) and s-p-OLE-10-GFP (green; not associated with LDs). TEM revealed that LDs in cells with s-p-OLE-10-GFP but not in cells with OLE-GFP were associated with PSVs (FIG. 4e), which agrees with CLSM findings that the s-p-10-OLE-GFP-associated-LDs were associated with PSVs.

Oleosin has a Mushroom- or T-Shaped Structure on the Surface of a LD.

Homology modeling was used to define the structure of oleosin on the surface of a LD (FIG. 5). The sequence of oleosins is unique, and no single template protein could be used for homology modeling. The OLE polypeptide was divided into segments and matched their sequence identities with segments of other proteins of known structures. The modeling template for the oleosin N-portion was the second transmembrane segment of phosphoserine aminotransferase of Mycobacterium tuberculosis; the two share 28% sequence identity. The modeling template for the oleosin C-portion was the third transmembrane segment of 6-amoinohexanoate cyclic dimer hydrolase of Arthrobacter species; the two share 30% sequence identity. Homology modeling predicted the oleosin N- and C-portions to be α-helices and random coils interacting with the LD surface PLs (panel b in FIG. 5). The model template for the oleosin central hydrophobic portion was 2 transmembrane segments linked by a proline-loop (18+17-loop+19 residues) of the alpha-1 subunit of human glycine receptor; the two share 38% sequence identity. Homology modeling predicted the oleosin central portion to be a hairpin of largely α-helix and partly random coil (panel b in FIG. 5). The 2 arms of the hairpin would interact for extra stability in the LD matrix. The loop possesses a 12-residue peptide of PX₅SPX₃P (SEQ ID NO:1), whose 3 proline and 1 serine residues are completely conserved among all oleosins of diverse plant species. No template peptide in proteins of known structures in other organisms has a sequence closely related to PX₅SPX₃P (SEQ ID NO:1) and its adjacent residues. It is believed that the loop has the 2 proline (P66, P70) and 1 serine (S65) residues interacting among themselves (panel b), with the third proline residue (P59) constituting the turn of the loop. The hydroxyl group of S65 could form a hydrogen bond with the hairpin peptide bond atoms, other serine and threonine residues adjacent to the loop, other serine and threonine residues in adjacent oleosin molecules, or the ester bond atoms of TAGs. The oleosin hairpin is ˜6 nm long assuming no bending, and thus is substantially longer than the ˜2-nm acyl moieties of a single PL layer on a LD or the ˜4-nm acyl moieties of a double PL layer of the ER membrane.

Overall, the oleosin structure is predicted to be a T- or mushroom-shaped molecule with the hairpin inserted into the TAG matrix of a LD (panel b in FIG. 5). This structure accommodates and allows compromises of different existing structural models of oleosin (see Introduction). The ˜6-nm hairpin without bending is stable in the LD matrix but unstable in the acyl leaflets of the ER membrane.

The length of the hairpin arms was artificially reduced from 30+12+26 residues (first arm+loop+second arm) to 10+12+10 residues and repeated the homology modeling, the hairpin arms became shorter (lower panel b in FIG. 5) and could be stable in the 2 acyl leaflets of the ER membrane.

Further Observation and Confirmation of Redistribution of Lipid Droplets (LDs) by Modified Oleosin: LDs are Directed to Endoplasmic Reticulum (ER) and/or Vacuole Instead of Cytosol.

Cells that were treated with digitonin (a mild detergent that breaks the plasma membrane but not the ER membrane) and a commercial protease retained the oleosin-LDs inside the ER lumen. Cells that were further treated with Triton-X (a stronger detergent that breaks also the ER membrane) had the oleosin-LDs proteolyzed (i.e., the ER membrane was broken, allowing the applied protease to enter the ER lumen and hydrolyze the oleosin-LDs).

### Conclusion

In addition to delineating the mechanism of oleosin and LD biosynthesis in plant cells, the current study demonstrates a successful redirection of massive LDs originally designated for cytosol to the ER lumen via 3 manipulations: (i) addition of a N-terminal ER targeting signal to oleosin, (ii) reduction of the oleosin hairpin length, and (iii) lack of abundant pre-existing native oleosins that would have already extracted ER-budding LDs to the cytosolic side or into cytosol. Further addition of a PSV-targeting propeptide to the modified oleosin transports the ER luminal LDs to PSVs and then large vacuoles in transformed tobacco cells. Without this PSV-targeting propeptide, LDs coated with the recombinant oleosin in the ER lumen did not move to the cellular exterior via a default pathway. This observation may reflect an undefined signal within proteins that could allow for secretion in tobacco cells; this signal peptide is absent in oleosin. However, in cells of other organisms, LDs coated with modified oleosin in the ER lumen may move to the cell exterior by default.

The current work has potential applications in various areas. Earlier studies have shown that after gene transformation and expression, oleosin is correctly targeted to LDs in yeast (37) and mammal (38) cells. Photosynthetic microbes are being used to produce oils in LDs as renewable biodiesels and high-value products. This industrial production is inefficient, because the microbes must be stressed (thereby stopping growth) to induce LD accumulation and then be killed to extract the LD oils. The photosynthetic microbes could be manipulated to excrete LDs, such that there is no need to stress and then kill the cells. Even if the LDs cannot be excreted but rather are stored in metabolically inert vacuoles, the compartmentation would eliminate metabolic feedback and allow for the continuous synthesis and accumulation of more oils (end metabolite). This can benefit agricultural production of oils in seeds and industrial use of yeast and other microbes to produce high-value lipid-related metabolites in LDs. For obesity treatments, the addition of an apparently inert recombinant oleosin to mammalian cells could lead to the transfer of cytosol-designated LDs to the intracellular secretory pathway for excretion. The present inventors have demonstrated that cytosol-designated LDs can be redirected to the ER lumen, PSVs and then large vacuoles in tobacco cells. Procedures for moving ER-luminal LDs to the cell exterior in plants and other organisms can be explored.

### Materials and Methods

Plant Materials.

The gametophyte of Physcomitrella patens subsp. patens were grown axenically on a solid Knop's medium supplemented with micronutrients (30) at 25±1° C. under a 16-h light (60˜100 μE m⁻²S⁻¹)/8-h dark cycle. Nicotiana tabaccum BY2 cell line was maintained as described (39).

Transient Expression with Physcomitrella Cells.

Expression constructs encoding OLE and recombinant OLEs (Table 1) and the primers (Table 2) are shown in Supplemental Data. The coding fragments were digested with BamHI and cloned into the expression site of a GFP expression vector (40) or an RFP expression vector (41) driven by a CaMV 35S promoter. A BIP-RFP expression vector of a similar construct (33) was also used. Transformation involved particle bombardment (30). Gold particles of 1.6-nm diameter coated with 5 μg plasmid DNA were bombarded with 900 psi under 28-in Hg vacuum onto 60-day-old leafy tissue from a distance of 6 cm in PDS-1000 (BIO-RAD, Hercules, Calif.). The bombarded tissue was observed with CLSM (Zeiss 510M for Physcomitrella, and Leica SP5 for tobacco) at time intervals. GFP and RFP were excited at 488 and 543 nm, and emission was detected at 500-530 and 565-615 nm, respectively.

Transformation of Tobacco BY-2 Cells.

Agrobacterium-mediated transformation of BY2 cells was as described (39). The expression vectors are shown in Table 1. Agrobacterium tumefaciens (strain GV3101) with the binary expression vector (100 μL) at OD₆₀₀ ˜0.5 were added to 4 ml of 3-d-old suspension cells. After co-cultivation at 25° C. for 2 d, cells were collected by centrifugation at 500 g for 2 min, washed 3 times with liquid medium containing 500 mg·L⁻¹ carbencillin, and transferred to solid BY-2 medium containing 500 mg·L⁻¹ carbencillin and selection antibiotic, 50 mg·L⁻¹ kanamycin and/or 20 mg·L⁻¹ hygromycin B. Expression was observed with CLSM.

Staining of LDs and ER.

LDs were stained with Nile Red (42). ER was stained with ER-Tracker-Red (BODIPY TR Glibenclamide, Invitrogen, Carlsbad, Calif.). Tissue was placed in a solution containing Nile Red stock (100 mg/ml DMSO) or ER-Tracker-Red stock (100 μg/110 μl DMSO) diluted 100× with 1× phosphate buffered saline (PBS: 10 mM K phosphate, pH 7.4, 138 mM NaCl and 2.7 mM KCl) for 10 min, washed with PBS twice, and observed with CLSM. Nile Red and ER-Tracker-Red were excited at 543 and 594 nm, and emission was detected at 565-615 and 610-650 nm, respectively.

Fluorescence Protease Protection Assay.

The assay was modified from Lorenz et al. (34). Physcomitrella cells were transformed with DNA constructs encoding s-OLE or s-OLE-10 (attached to GFP or RFP) and BIP-RFP. After 12 h, cells were incubated in 1×PBS for 10 min, washed with PBS twice, and permeated with 25 μg/mL digitonin for 10 min. Then, 4-mM trypsin in PBS was added, and digestion was allowed for 20 min. Fluorescence was observed with CLSM before and after trypsin treatment.

Electron Microscopy.

Tissue was fixed via high-pressure freezing or chemical fixation. For freezing fixation, tissue was fixed in a high-pressure freezer (Leica EM PACT2) and then subjected to freeze substitution in ethanol containing 0.2% glutaraldehyde and 0.1% uranyl acetate in Leica AFS System and embedded in LR Gold resin (Structural Probe, West Chester, Pa.). For chemical fixation, tissue was fixed with 2.5% glutaraldehyde, 4% paraformaldehyde and 0.1 M K-phosphate (pH 7.0) at 4° C. for 24 h. Materials were washed with 0.1 M K-phosphate buffer (pH 7.0) for 10 min twice and treated with 1% 0504 in 0.1 M K-phosphate (pH 7.0) at 24° C. for 4 h. Fixed materials were rinsed with 0.1 M K-phosphate buffer (pH 7.0), dehydrated in an acetone series and embedded in Spurr resin. Ultrathin sections (70-90 nm) were stained with uranyl acetate and lead citrate and examined with a Philips CM 100 TEM at 80 KV.

All patents, patent applications, and other publications, including GenBank Accession Numbers, cited in this application are incorporated by reference in the entirety for all purposes.

TABLE 1
Information on DNA expression constructs

| Expression | | | Insertion | | |
|---|---|---|---|---|---|
| proteins | Expression cell | Vector backbone | site | Primer No. | References |
| GFP | Physcomitrella | HBT-sGFP(S65T)-NOS | | | 2 |
| OLE-GFP | Physcomitrella | HBT-sGFP(S65T)-NOS | BamHI | 1, 2 | This report |
| OLEΔC-GFP | Physcomitrella | HBT-sGFP(S65T)-NOS | BamHI | 1, 4 | This report |
| OLEΔN31-GFP | Physcomitrella | HBT-sGFP(S65T)-NOS | BamHI | 2, 3 | This report |
| OLE-15-GFP | Physcomitrella | HBT-sGFP(S65T)-NOS | BamHI | 1, 2, 5, 6 | This report |
| OLE-10-GFP | Physcomitrella | HBT-sGFP(S65T)-NOS | BamHI | 1, 2, 7, 8 | This report |
| OLE-5-GFP | Physcomitrella | HBT-sGFP(S65T)-NOS | BamHI | 1, 2, 9, 10 | This report |
| OLE-PPPP-GFP | Physcomitrella | HBT-sGFP(S65T)-NOS | BamHI | 1, 2, 11, 12 | This report |
| OLE-SSSS-GFP | Physcomitrella | HBT-sGFP(S65T)-NOS | BamHI | 1, 2, 19-22 | This report |
| OLE-PYPP-GFP | Physcomitrella | HBT-sGFP(S65T)-NOS | BamHI | 1, 2, 13, 14 | This report |
| OLE-LSLL-GFP | Physcomitrella | HBT-sGFP(S65T)-NOS | BamHI | 1, 2, 15-18 | This report |
| s-OLE-GFP | Physcomitrella | HBT-sGFP(S65T)-NOS | BamHI | 1, 2, 24, 25 | This report |
| s-OLE15-GFP | Physcomitrella | HBT-sGFP(S65T)-NOS | BamHI | 2, 24 | This report |
| s-OLE10-GFP | Physcomitrella | HBT-sGFP(S65T)-NOS | BamHI | 2, 24 | This report |
| s-OLE5-GFP | Physcomitrella | HBT-sGFP(S65T)-NOS | BamHI | 2, 24 | This report |
| s-OLE10-RFP | Physcomitrella | pUC/326RFP | BamHI | 24, 26 | 3 |
| BIP-RFP | Physcomitrella | pUC/326RFP | | | 4 |
| OLE-GFP | Tobacco | pCAMBIA1302 | Gateway | 28, 30 | This report |
| s-OLE10-GFP | Tobacco | pCAMBIA1302 | Gateway | 27, 28, 29 | This report |
| s-L-OLE10-GFP | Tobacco | pK2GW7 | Gateway | 38, 39, 40 | This report |
| OLEΔN24-GFP | Tobacco | pK2GW7 | Gateway | 35, 38 | This report |
| OLEΔN28-GFP | Tobacco | pK2GW7 | Gateway | 36, 38 | This report |
| OLEΔN31-GFP | Tobacco | pK2GW7 | Gateway | 37, 38 | This report |
| AtT-GFP | Tobacco | pK2GW7 | Gateway | 31, 34, 39 | This report |
| AtTΔN6-GFP | Tobacco | pK2GW7 | Gateway | 32, 34, 39 | This report |
| AtTΔN10-GFP | Tobacco | pK2GW7 | Gateway | 33, 34, 39 | This report |
| s-P-RFP | Tobacco | pCAMBIA1300MCS | Xbal, Sacl | | From Dr. N. Raikhel |

TABLE 2
Information on primers

| Primer | | | SEQ |
|---|---|---|---|
| No. | Primer name | Sequence 5′→3′ | ID NO: |
| 1 | ppole1_BamH1F | ATCGGGATCCATGGATAATGCCAAAACC | 3 |
| 2 | ppole1_BamH1R | AGCTGGATCCAGACAAGTATACCCCGAAGG | 4 |
| 3 | ole1hpBamH1F | ATGCGGATCCATCCTCGTCGCGGTGG | 5 |
| 4 | 01e1hpBamH1R | ATGCGGATCCTTTGTACACCCAGACAGCG | 6 |
| 5 | ole1N3′hp15a5′R | CCAGTCAGCGTGAGGCCGATGGTAACCAATCCTAGCA | 7 |
| 6 | ole1hp15a3′C5′F | GCAGCCTGTTGGGTTTCAAATACTACAAGGGTGGTCAC | 8 |
| 7 | ole1N3′hp10a5′R | AGCCAATGGTGGTGCCGATGGTAACCAATCCTAGCA | 9 |
| 8 | ole1hp10a3′C5′F | CGTTTTTCGCTATCAGCAAATACTACAAGGGTGGTCAC | 10 |
| 9 | o1e1N3′hp5a5′R | GGGCAATGACAGAAAGAAGATGGTAACCAATCCTAGCA | 11 |
| 10 | ole1hp5a3′C5′F | GCTGGCAATTTTTGCGAAATACTACAAGGGTGGTCAC | 12 |
| 11 | ole1 1PF | CCGTGCTCATTTTCTTCCCCCCTATTCTCGTCCCG | 13 |
| 12 | ole1 1PR | CGGGACGAGAATAGGGGGGAAGAAAATGAGCACGG | 14 |
| 13 | ole1 1YF | CCGTGCTCATTTTCTTCTACCCTATTCTCGTCCCG | 15 |
| 14 | ole1 1YR | CGGGACGAGAATAGGGTAGAAGAAAATGAGCACGG | 16 |
| 15 | ole1 1LF | CTTTCTGTCATTGCTCGTGCTCATTTTCTTCAGCCC | 17 |
| 16 | ole1 1LR | GGGCTGAAGAAAATGAGCACGAGCAATGACAGAAAG | 18 |
| 17 | ole1 2LF | CATTTTCTTCAGCCTTATTCTCGTCCTGCTGGCAATTTTTGCG | 19 |
| 18 | ole1 2LR | CGCAAAAATTGCCAGCAGGACGAGAATAAGGCTGAAGAAAATG | 20 |
| 19 | ole1 1SF | CTTTCTGTCATTGTCCGTGCTCATTTTCTTCAGCCC | 21 |
| 20 | o1e1 1SR | GGGCTGAAGAAAATGAGCACGGACAATGACAGAAAG | 22 |
| 21 | o1e1 2SF | CATTTTCTTCAGCTCTATTCTCGTCTCGCTGGCAATTTTTGCG | 23 |
| 22 | o1e1 25R | CGCAAAAATTGCCAGCGAGACGAGAATAGAGCTGAAGAAAATG | 24 |
| 23 | ole1 1LF | CTTTCTGTCATTGCTCGTGCTCATTTTCTTCAGCCC | 25 |
| 24 | ASP_BamHI | ATCGGGATCCATGGGGGCATCGAGGAGT | 26 |
| 25 | ASPr_OLE | TGGTTTTGGCATTATCCATTGCCTCAGCTAAGGCTGC | 27 |
| 26 | ole-BamH1R_RFP | AGCTGGATCCAGACAAGTATACCCCGAAGG | 28 |
| 27 | Atclv3spF_NcoI | CATGCCATGGATTCGAAGAGTTTTCTG | 29 |
| 28 | OLE1RBglII | GGAAGATCTAGACAAGTATACCCCGAAGGAC | 30 |
| 29 | Atclv3spR_ppOLE1 | GCCTTGGTTTTGGCATTATCATCAGAAGCATCATGAAGGAAC | 31 |
| 30 | ole1F_NcoI | CATGCCATGGATAATGCCAAAACC | 32 |
| 31 | AtTGatF | GGGGACAAGTTTGTACAAAAAAGCAGGCTATGTTTGAGATTATTCAGGCGGTC | 33 |
| 32 | AtTN1GatF | GGGGACAAGTTTGTACAAAAAAGCAGGCTATGGCGGTCTTCTCCGCCGGG | 34 |
| 33 | AtTN2GatF | GGGGACAAGTTTGTACAAAAAAGCAGGCTATGGCCGGGGTTGCACTAGCTC | 35 |
| 34 | TR_GFP | CTCGCCCTTGCTCACCATGACGCCGGAACCTGCTGG | 36 |
| 35 | OLEN2GatF | GGGGACAAGTTTGTACAAAAAAGCAGGCTATGAGGCAGGTGCTAGGATTGGTTAC | 37 |
| 36 | OLEN3GatF | GGGGACAAGTTTGTACAAAAAAGCAGGCTATGTTGGTTACCATCCTCGTCGCG | 38 |
| 37 | OLEN4GatF | GGGGACAAGTTTGTACAAAAAAGCAGGCTATGCTCGTCGCGGTGGGTACTGTC | 39 |
| 38 | sGFPR_GatR | GGGGACCACTTTGTACAAGAAAGCTGGGTTTACTTGTACAGCTCGTCCATG | 40 |
| 39 | phaseolinSP_GatF | GGGGACAAGTTTGTACAAAAAAGCAGGCTATGATGAGAGCAAGGGTTCCACTCC | 41 |
| 40 | propeptide_OLE1R | GCCTTGGTTTTGGCATTATCATTAAAATTTGGTACCACTGGC | 42 |

### REFERENCES

1. Huang A H C (1992) Oil bodies and oleosins in seeds. Annu Rev Plant Physiol Mol Biol 43:177-200.
2. Huang A H C (2018) Plant lipid droplets and their associated proteins: potential for rapid advances. Plant Physiol 176: 1894-1918.
3. Pyc M, Cai Y, Greer M S, Yurchenko O, Chapman K D, Dyer J M, Mullen R T (2017) Turing over a new leaf in lipid droplet biology. Trends in Plant Sci 22: 596-609.
4. Shimada T L, Takano Y, Shimada T, Fujiwara M, Fukao Y, Mori M, Okazaki Y, Saito K, Sasaki R, Aoki K, Hara-Nishimura I (2014) Leaf oil body functions as a subcellular factory for the production of a phytoalexin in Arabidopsis. Plant Physiol 164: 105-118.
5. Koch B, Schmidt C, Daum G (2014) Storage lipids of yeasts: a survey of nonpolar lipid metabolism in Saccharomyces cerevisiae, Pichia pastoris, and Yarrowia lipolytica. FEMS Microbiol Rev 38: 892-915.
6. Kory N, Farese Jr R V, Walther T C (2016) Targeting fat: mechanisms of protein localization to lipid droplets. Trends in Cell Biol 26: 535-546.
7. Barbosa A D, Siniossoglou S (2017) Function of lipid droplet-organelle interactions in lipid homoestasis. Biochim Biophys Acta-Mol Cell Res 1864: 1459-1468.
8. Barneda D, Christian M (2017) Lipid droplet growth: regulation of a dynamic organelle. Current Opinion in Cell Biol 47: 9-15.
9. Chen X, Goodman J M (2017) The collaborative work of droplet assembly. Biochim Biophys Acta-Mol Cell Biol Lipids 1862: 1205-1211.
10. Walther T C, Chung J, Farese R V (2017) Lipid droplet biogenesis. Ann Rev Cell Devel Biol 33: 491-510
11. Taparia T, Manjari MVSS, Mehrotra R, Shukla P, Mehdotra A (2016) Developments and challenges in biodiesel production from microalgae: A review. Biotech Appl Biochem 63: 715-726.
12. Matos A P (2017) The impact of microalgae in food science and technology. J Am Oil Chem Soc. 94: 1333-1350.
13. Tzen J T & Huang A H C (1992) Surface structure and properties of plant seed oil bodies. J Cell Biol 117(2):327-335.
14. Lee W S. Tzen J T C, Kridl J C, Radke S E, Huang A H C. 1991. Maize oleosin is correctly targeted to seed oil bodies in Brassica napus transformed with the maize oleosin gene. Proc. Nat. Acad. Sci. 88(14): 6181-6185.
15. Alexander L G, et al. (2002) Characterization and modelling of the hydrophobic domain of a sunflower oleosin. Planta 214(4):546-551.
16. Li M, et al. (2002) Purification and structural characterization of the central hydrophobic domain of oleosin. J Biol Chem 277(40):37888-37895.
17. Cao Cao Y Z, Huang A H C (1986) Diacylglycerol acyltransferase in maturing oil seeds of maize and other species. Plant Physiol 82: 813-820
18. Hsieh K & Huang A H C (2007) Tapetosomes in Brassica tapetum accumulate endoplasmic reticulum-derived flavonoids and alkanes for delivery to the pollen surface. Plant cell 19(2):582-596.
19. Lacey D J, Wellner N, Beaudoin F, Napier J A, & Shewry P R (1998) Secondary structure of oleosins in oil bodies isolated from seeds of safflower (Carthamus tinctorius L.) and sunflower (Helianthus annuus L.). Biochem J 334(Pt 2):469-477.
20. Shockey J M, et al. (2006) Tung tree DGAT1 and DGAT2 have nonredundant functions in triacylglycerol biosynthesis and are localized to different subdomains of the endoplasmic reticulum. Plant cell 18(9):2294-2313.
21. Abell B M, High S, & Moloney M M (2002) Membrane protein topology of oleosin is constrained by its long hydrophobic domain. J Biol Chem 277(10):8602-8610.
22. Beaudoin F & Napier J A (2002) Targeting and membrane-insertion of a sunflower oleosin in vitro and in Saccharomyces cerevisiae: the central hydrophobic domain contains more than one signal sequence, and directs oleosin insertion into the endoplasmic reticulum membrane using a signal anchor sequence mechanism. Planta 215(2):293-303.
23. Thoyts P J, et al. (1995) Expression and in vitro targeting of a sunflower oleosin. Plant Mol Biol 29(2):403-410.
24. Beaudoin F, Wilkinson B M, Stirling C J, & Napier J A (2000) In vivo targeting of a sunflower oil body protein in yeast secretory (sec) mutants. Plant J 23(2):159-170.
25. van Rooijen G J & Moloney M M (1995) Structural requirements of oleosin domains for subcellular targeting to the oil body. Plant Physiol 109(4):1353-1361.
26. Martinoia E, Meyer S, De Angeli A, & Nagy R (2012) Vacuolar transporters in their physiological context. Annu Rev Plant Biol 63:183-213.
27. Zhang C, Hicks G R, & Raikhel N V (2014) Plant vacuole morphology and vacuolar trafficking. Front Plant Sci 5:476.
28. Barrieu F & Chrispeels M J (1999) Delivery of a secreted soluble protein to the vacuole via a membrane anchor. Plant Physiol 120(4):961-968.
29. Huang C Y, Huang A H C (2017) Unique motifs and length of hairpin in oleosin targets the cytosolic side of endoplasmic reticulum and budding lipid droplets. Plant Physiol 174: 2248-2260.
30. Huang C Y, Chung C I, Lin Y C, Hsing Y I, & Huang A H C (2009) Oil bodies and oleosins in Physcomitrella possess characteristics representative of early trends in evolution. Plant Physiol 150(3):1192-1203.
31. Abell B M, Hahn M, Holbrook L A, & Moloney M M (2004) Membrane topology and sequence requirements for oil body targeting of oleosin. Plant J 37(4):461-470.
32. Abell B M, et al. (1997) Role of the proline knot motif in oleosin endoplasmic reticulum topology and oil body targeting. Plant cell 9(8):1481-1493.
33. Marella H H, Sakata Y, & Quatrano R S (2006) Characterization and functional analysis of ABSCISIC ACID INSENSITIVE 3-like genes from Physcomitrella patens. Plant J 46(6):1032-1044.
34. Lorenz H, Hailey D W, & Lippincott-Schwartz J (2006) Fluorescence protease protection of GFP chimeras to reveal protein topology and subcellular localization. Nat Methods 3(3):205-210.
35. Kim D H, et al. (2001) Trafficking of phosphatidylinositol 3-phosphate from the trans-Golgi network to the lumen of the central vacuole in plant cells. Plant cell 13(2):287-301.
36. Frigerio L, et al. (2001) The internal propeptide of the ricin precursor carries a sequence-specific determinant for vacuolar sorting. Plant Physiol 126(1):167-175.
37. Ting J T, Balsamo R A, Ratnayake C, & Huang A H C (1997) Oleosin of plant seed oil bodies is correctly targeted to the lipid bodies in transformed yeast. J Biol Chem 272 (6):3699-3706.
38. Hope R G, Murphy D J, & McLauchlan J (2002) The domains required to direct core proteins of hepatitis C virus and GB virus-B to lipid droplets share common features with plant oleosin proteins. J Biol Chem 277(6):4261-4270.
39. Brandizzi F, Irons S, Kearns A, & Hawes C (2003) BY-2 cells: culture and transformation for live cell imaging. Curr Protoc Cell Biol/editorial board, Juan S B et al. Chapter 1:Unit 1 7.
40. Chiu W, et al. (1996) Engineered GFP as a vital reporter in plants. Curr Biol 6(3):325-330.
41. Lee Y J, Kim D H, Kim Y W, & Hwang I (2001) Identification of a signal that distinguishes between the chloroplast outer envelope membrane and the endomembrane system in vivo. Plant cell 13(10):2175-2190.
42. Huang C Y, Chen P Y, Huang M D, Tsou C H, Jane W N, Huang A H C. 2013. Tandem oleosin genes in a cluster acquired in Brassicaceae created tapetosomes and conferred additive benefit of pollen vigor. Proc. Natl. Acad. Sci. 110(35): 14480-14485.

1. A modified oleosin protein generated from a native oleosin protein comprising an amphipathic N-terminal peptide, an amphipathic C-terminal peptide, and a hairpin in the middle comprising a hairpin loop flanked by two hairpin arms, wherein the modified oleosin protein comprises (1) an endoplasmic reticulum (ER)-targeting peptide at the N-terminus of the modified oleosin protein; and (2) one or both truncated hairpin arms of the native oleosin protein.

2. The modified oleosin protein of claim 1, further comprising a vacuole-targeting sequence.

3. The modified oleosin protein of claim 2, wherein the vacuole-targeting sequence is a protein storage vacuoles (PSV)-targeting sequence.

4. The modified oleosin protein of claim 2, wherein the vacuole-targeting sequence is located between the ER-targeting peptide and the truncated hairpin arm.

5. The modified oleosin protein of claim 1, wherein the one or both truncated hairpin arms are left with 5-15 amino acids.

6. The modified oleosin protein of claim 1, wherein the amphipathic N- or C-terminal peptide of the native oleosin protein is truncated or deleted.

7. The modified oleosin protein of claim 1, which includes the initial 5-10 amino acids of the first hairpin arm.

8. The modified oleosin protein of claim 1, further comprising an additional heterologous peptide.

9. The method of claim 8, wherein the heterologous peptide is a green fluorescent protein (GFP) or β-glucuronidase (GUS).

10. The modified oleosin protein of claim 1, which is covalently linked to a detectable label.

11. An isolated polynucleotide sequence encoding the modified oleosin protein of claim 1.

12. An expression cassette comprising the polynucleotide sequence of claim 11.

13. A vector comprising the polynucleotide sequence of claim 11.

14. A host cell comprising the expression cassette of claim 12.

15. The host cell of claim 14, which is a plant cell, an animal cell, a fungal cell, or a bacterial cell.

16. The host cell of claim 14, which has its genomic sequence altered to express no or a reduced level of endogenous oleosin protein.

17. An organism comprising the cell of claim 14.

18. The organism of claim 17, which is a plant, an animal, or a microbe.

19. A method of regulating distribution of subcellular lipid within a cell, comprising introducing the polynucleotide of claim 11 into the cell.

20. A method for promoting secretion of lipids from within a cell, the method comprising introducing the polynucleotide of claim 11 into the cell.

21. The method of claim 19, wherein the modified oleosin protein is expressed in the cell permanently.

22. The method of claim 19, wherein the modified oleosin protein is expressed in the cell transiently.

23. The method of claim 19, wherein the cell has its genomic sequence altered to express no or a reduce level of endogenous oleosin protein.

24. The method of claim 19, wherein the cell is within a living organism.

25. The method of claim 24, wherein the living organism is a plant, an animal, or a microbe.

26. A method for generating a cell with increased extracellular secretion of lipids, the method comprising introducing the polynucleotide of claim 11 into the cell.

27. The method of claim 26, further comprising a step of selecting a cell, subsequent to the introducing step, for increased extracellular secretion of lipids.

28. The method of claim 27, further comprising a step of collecting secreted lipids from a cell exhibiting increased extracellular secretion of lipids.

29. The method of claim 26, wherein the cell has its genomic sequence altered to express no or a reduce level of endogenous oleosin protein.

30. The method of claim 26, wherein the cell is within a living organism.

31. An organism generated by the method of claim 29.

32. The organism of claim 30, which is a plant, a non-human animal, or a microbe.


SEQUENCE LISTING (42 sequences)

SEQ ID NO: 1 - protein, 12 aa, Artificial Sequence
  Synthetic construct
  MISC_FEATURE (2)..(6): Xaa = any nonpolar amino acid
  MISC_FEATURE (9)..(11): Xaa = any nonpolar amino acid
PXXXXXSPXXXP

SEQ ID NO: 2 - protein, 146 aa, Physcomitrella patens
MDNAKTKANDLYYRAQQNAPLNRRQVLGLVTILVAVGTVITIAGLTLTGTTIGFFLSLPVLIFFSPILVPLAIFAFFAISSLLGFGGFVSFAVWVYKGGHPVGSDQVDAARYRIAEGAKEVKDRTYAAARDVQGYVASKTDTSVAA

SEQ ID NO: 3 - DNA, 28 bp, Artificial Sequence
  Synthetic construct
atcgggatccatggataatgccaaaacc

SEQ ID NO: 4 - DNA, 30 bp, Artificial Sequence
  Synthetic construct
agctggatccagacaagtataccccgaagg

SEQ ID NO: 5 - DNA, 26 bp, Artificial Sequence
  Synthetic construct
atgcggatccatcctcgtcgcggtgg

SEQ ID NO: 6 - DNA, 29 bp, Artificial Sequence
  Synthetic construct
atgcggatcctttgtacacccagacagcg

SEQ ID NO: 7 - DNA, 37 bp, Artificial Sequence
  Synthetic construct
ccagtcagcgtgaggccgatggtaaccaatcctagca

SEQ ID NO: 8 - DNA, 38 bp, Artificial Sequence
  Synthetic construct
gcagcctgttgggtttcaaatactacaagggtggtcac

SEQ ID NO: 9 - DNA, 36 bp, Artificial Sequence
  Synthetic construct
agccaatggtggtgccgatggtaaccaatcctagca

SEQ ID NO: 10 - DNA, 38 bp, Artificial Sequence
  Synthetic construct
cgtttttcgctatcagcaaatactacaagggtggtcac

SEQ ID NO: 11 - DNA, 38 bp, Artificial Sequence
  Synthetic construct
gggcaatgacagaaagaagatggtaaccaatcctagca

SEQ ID NO: 12 - DNA, 37 bp, Artificial Sequence
  Synthetic construct
gctggcaatttttgcgaaatactacaagggtggtcac

SEQ ID NO: 13 - DNA, 35 bp, Artificial Sequence
  Synthetic construct
ccgtgctcattttcttcccccctattctcgtcccg

SEQ ID NO: 14 - DNA, 35 bp, Artificial Sequence
  Synthetic construct
cgggacgagaataggggggaagaaaatgagcacgg

SEQ ID NO: 15 - DNA, 35 bp, Artificial Sequence
  Synthetic construct
ccgtgctcattttcttctaccctattctcgtcccg

SEQ ID NO: 16 - DNA, 35 bp, Artificial Sequence
  Synthetic construct
cgggacgagaatagggtagaagaaaatgagcacgg

SEQ ID NO: 17 - DNA, 36 bp, Artificial Sequence
  Synthetic construct
ctttctgtcattgctcgtgctcattttcttcagccc

SEQ ID NO: 18 - DNA, 36 bp, Artificial Sequence
  Synthetic construct
gggctgaagaaaatgagcacgagcaatgacagaaag

SEQ ID NO: 19 - DNA, 43 bp, Artificial Sequence
  Synthetic construct
cattttcttcagccttattctcgtcctgctggcaatttttgcg

SEQ ID NO: 20 - DNA, 43 bp, Artificial Sequence
  Synthetic construct
cgcaaaaattgccagcaggacgagaataaggctgaagaaaatg

SEQ ID NO: 21 - DNA, 36 bp, Artificial Sequence
  Synthetic construct
ctttctgtcattgtccgtgctcattttcttcagccc

SEQ ID NO: 22 - DNA, 36 bp, Artificial Sequence
  Synthetic construct
gggctgaagaaaatgagcacggacaatgacagaaag

SEQ ID NO: 23 - DNA, 43 bp, Artificial Sequence
  Synthetic construct
cattttcttcagctctattctcgtctcgctggcaatttttgcg

SEQ ID NO: 24 - DNA, 43 bp, Artificial Sequence
  Synthetic construct
cgcaaaaattgccagcgagacgagaatagagctgaagaaaatg

SEQ ID NO: 25 - DNA, 36 bp, Artificial Sequence; identical to SEQ ID NO: 17
  Synthetic construct

SEQ ID NO: 26 - DNA, 28 bp, Artificial Sequence
  Synthetic construct
atcgggatccatgggggcatcgaggagt

SEQ ID NO: 27 - DNA, 37 bp, Artificial Sequence
  Synthetic construct
tggttttggcattatccattgcctcagctaaggctgc

SEQ ID NO: 28 - DNA, 30 bp, Artificial Sequence; identical to SEQ ID NO: 4
  Synthetic construct

SEQ ID NO: 29 - DNA, 27 bp, Artificial Sequence
  Synthetic construct
catgccatggattcgaagagttttctg

SEQ ID NO: 30 - DNA, 31 bp, Artificial Sequence
  Synthetic construct
ggaagatctagacaagtataccccgaaggac

SEQ ID NO: 31 - DNA, 42 bp, Artificial Sequence
  Synthetic construct
gccttggttttggcattatcatcagaagcatcatgaaggaac

SEQ ID NO: 32 - DNA, 24 bp, Artificial Sequence
  Synthetic construct
catgccatggataatgccaaaacc

SEQ ID NO: 33 - DNA, 53 bp, Artificial Sequence
  Synthetic construct
ggggacaagtttgtacaaaaaagcaggctatgtttgagattattcaggcggtc

SEQ ID NO: 34 - DNA, 50 bp, Artificial Sequence
  Synthetic construct
ggggacaagtttgtacaaaaaagcaggctatggcggtcttctccgccggg

SEQ ID NO: 35 - DNA, 51 bp, Artificial Sequence
  Synthetic construct
ggggacaagtttgtacaaaaaagcaggctatggccggggttgcactagctc

SEQ ID NO: 36 - DNA, 36 bp, Artificial Sequence
  Synthetic construct
ctcgcccttgctcaccatgacgccggaacctgctgg

SEQ ID NO: 37 - DNA, 55 bp, Artificial Sequence
  Synthetic construct
ggggacaagtttgtacaaaaaagcaggctatgaggcaggtgctaggattggttac

SEQ ID NO: 38 - DNA, 53 bp, Artificial Sequence
  Synthetic construct
ggggacaagtttgtacaaaaaagcaggctatgttggttaccatcctcgtcgcg

SEQ ID NO: 39 - DNA, 53 bp, Artificial Sequence
  Synthetic construct
ggggacaagtttgtacaaaaaagcaggctatgctcgtcgcggtgggtactgtc

SEQ ID NO: 40 - DNA, 51 bp, Artificial Sequence
  Synthetic construct
ggggaccactttgtacaagaaagctgggtttacttgtacagctcgtccatg

SEQ ID NO: 41 - DNA, 54 bp, Artificial Sequence
  Synthetic construct
ggggacaagtttgtacaaaaaagcaggctatgatgagagcaagggttccactcc

SEQ ID NO: 42 - DNA, 42 bp, Artificial Sequence
  Synthetic construct
gccttggttttggcattatcattaaaatttggtaccactggc